(19) United States
(12) Patent Application Publication
(10) Pub. No.: US 2007/0197887 A1
(43) Pub. Date: Aug. 23, 2007

(54) Noninvasive vital signs sensor

(75) Inventors: Donna R. Lunak, Saint Paul, MN (US); Timothy J. O'Malley, Topsfield, MA (US)

Correspondence Address: ALTERA LAW GROUP, LLC, 6500 CITY WEST PARKWAY, SUITE 100, MINNEAPOLIS, MN, 55344-7704, US

(73) Assignee: Medwave, Inc.

(21) Appl. No.: 11/357,877

(22) Filed: Feb. 17, 2006

(51) Int. Cl. A61B 5/00

(52) U.S. Cl. 600/323; 600/344; 600/324

(57) ABSTRACT

A noninvasive vital signs monitoring device uses a sensor which is capable of providing data for calculating pulse rate, blood pressure (for example, systolic, diastolic, and/or mean pressure) and blood oxygen saturation. In some embodiments, the sensor is also capable of providing data for calculating tissue perfusion. Optionally, a temperature sensor may be included as well.

### BACKGROUND OF THE INVENTION

1. Field of the Invention

The present invention relates to acquisition of data for patient vital signs, and more particularly to sensors for acquiring data for patient vital signs.

2. Description of the Related Art

In many situations, including in medical facilities, in the home, and in emergency situations such as an accident scene, ambulance transport, and the emergency room, the monitoring of a patients vital signs, such as temperature, blood oxygen saturation, and blood pressure, is important. For proper care, it is important to monitor these vital signs over a period of time, so that any appropriate actions may be taken in response to events and trends in the vital signs.

A patient's body core temperature is typically measured via a probe placed in the inner ear, which responds to changes in core temperature more quickly than most other body parts. Electrical signals are delivered from the probe via one or more wires to a processor, typically located away from the probe (as opposed to located in close proximity to the ear). The processor converts the signals from the probe into a temperature value that may be read visually by the staff of the hospital. Additionally, the temperature values over a period of time may be stored or displayed by the processor, so that trends may be detected.

Blood oxygen saturation, commonly referred to as SpO₂, is measured by a pulse oximeter and represents the fraction of hemoglobin (Hb) in the blood “saturated” with oxygen. The pulse oximeter displays the fraction (as a percentage) of Hb with a bound oxygen molecule. Healthy individuals typically have blood oxygen saturation levels in the range of 95% or higher. Historically, pulse oximeters have taken the form of a finger-mounted device for adults and toe-mounted for newborns.

Pulse oximeters are opto-electronic devices typically with two light emitting diodes (LED's) radiating at separate wavelengths (normally in the range of 650 nm and 800 nm respectively) and a single photo detector. The LED outputs are partially absorbed by hemoglobin, by amounts which differ depending on whether the hemoglobin is saturated or desaturated with oxygen. By calculating the relative absorption at the two wavelengths, an algorithm can compute the fraction or percentage of hemoglobin which is oxygenated. The oximeter algorithm is dependant on a pulsatile flow, and is capable of distinguishing pulsatile flow from typically static signals such as tissue or venous signals to limit the respond to arterial flow.

Blood pressure is commonly measured noninvasively by the use of an oscillatory cuff. A cuff operates in accordance with either an oscillometric or ausculatory method. However, since the oscillometric and auscultatory methods require inflation of the cuff, these methods are not entirely suitable for performing frequent measurements and measurements over long periods of time. The frequency of measurement is limited by the time required to inflate and deflate the cuff, and the pressure imposed by the cuff is uncomfortable to the patient and occludes the artery, thereby affecting any “downstream” measurements such as blood oxygen saturation. Moreover, both the oscillometric and auscultatory methods lack accuracy and consistency. Another disadvantage of the cuff is that it must be made available in numerous sizes to accommodate different patients. Commonly cuffs are provided in six different sizes. Typically all of the different cuffs must be readily available to the practitioner, resulting in unnecessary effort for the practitioner. If the different cuff sizes are stored with the instrument, this unnecessarily increases the size of the storage case.

The cuff is also quite disadvantageous when used on morbidly obese patients. Regardless of how a cuff is sized for the patient, the cuff yields inaccurate results and tends to injure the soft tissues of the patient.

While blood pressure may be measured noninvasively using a cuff, a superior approach for the noninvasive monitoring of blood pressure applies a pressure sensor to the patient's wrist over the radial artery with a varying hold-down force, so that the sensor presses the artery against the radius bone. The sensor should be positioned at the distal edge of the radius bone. Devices of this type and their associated methods of calculating blood pressure are described in various patents, including the sensor described in U.S. Pat. No. 5,450,852 entitled “Continuous Non-Invasive Blood Pressure Monitoring System” which issued Sep. 19, 1995 to Archibald et al.; the basic algorithm described in U.S. Pat. No. 5,797,850 issued Aug. 25, 1998 to Archibald et al., the beat onset detection method as described in U.S. Pat. No. 5,720,292 issued Feb. 24, 1998 to Poliac, and the segmentation estimation method as described in U.S. Pat. No. 5,738,103 issued Apr. 14, 1998 to Poliac. Commercially available devices of the sensor-based type include the Vasotrac®) model AMP205A NIBP monitor system, which is available from Medwave Inc. of Danvers, Mass. Revision K of the Vasotrac monitor uses a manual motion compensation technique, while Revision L uses an automatic motion compensation technique.

The sensor-based type of device is advantageous over the cuff in many respects, being both accurate with a typical mean correlation of about 0.97 with a well managed arterial line, as well as being fast with the ability to calculate four accurate readings of systolic, diastolic, and mean pressure and heart rate per minute. Moreover, some versions of the device are able to store and display full pulse arterial waveforms. The sensor-based type of device is also convenient for the patient. Because the device uses a relatively small soft-surfaced sensor placed over the radial artery at the wrist, the patient does not experience the discomfort of a fully occluded artery and need not remove any clothing or roll his/her sleeve to the upper arm. Unlike other techniques such as the cuff, operation with the sensor-type device is smooth with little noise, so it generally does not disturb patients who are resting.

The sensor-based type of device has also been found to achieve significantly greater accuracy than the upper arm oscillometric cuff pressure monitoring. While pressure monitoring using the arterial canula is still the gold standard of blood pressure measurement, the sensor-based type of device should be a valuable tool for monitoring the blood pressure of morbidly obese patients perioperatively without the possible negative side effects of the arterial canula.

While temperature, blood oxygen saturation, and blood pressure measuring devices are widely available as separate systems, they have also been integrated into single systems generally known as vital signs monitors, and have also been integrated along with other measurements such as ECG into single systems known as bedside monitors. Such monitors are available from various manufactures, including Welch Allyn Inc. of Beaverton, Oreg., and Nihon Kohden America, Inc. of Foothill Ranch, Calif. The Vital Signs Monitor 300 Series available from Welch Allyn, for example, is configurable for noninvasively measuring blood pressure with a cuff, as well as pulse oximetry and temperature. No waveforms are displayed. The Vital Signs Monitor Model OPV1500 available from Nihon Kohden America, for example, noninvasively measures blood pressure with a cuff, and may also perform pulse oximetry and ECG measurements. The information displayed is a respiration number and an ECG waveform, an SpO₂ number and an SpO₂ waveform, and pulse rate, systolic pressure, diastolic pressure, and mean pressure numbers. An example of a full featured bedside monitor is the Procyon series monitor, available from Nihon Kohden America. The Procyon monitor can simultaneously accept the inputs from various devices designed to measure ECG/respiration, non-invasive blood pressure), BP, ETCO₂, FiO₂, temperature, and cardiac output. The configurable screen can display a plethora of information. However, inasmuch as cuffs do not provide pulse waveform information, none of these monitors can display pulse waveform information (as opposed to the heart's electrical activity as reported by an ECG) from which the mechanical activity of the patient's heart can be observed.

Another type of bedside monitor is the Model BSM-9510 bedside monitor, which is available from Nihon Kohden Corporation of Tokyo, Japan. The model BSM-9510 bedside monitor performs a great many different measurements, including the noninvasive measurement of blood pressure with a cuff. The monitor also features a modular design which accommodates a sensor-based noninvasive blood pressure monitor module such as the model MJ23 CNIBP OEM Module, which is available from Medwave Inc. of Danvers, Mass. The model BSM-9510 as equipped with the model MJ23 CNIBP OEM module is able to display pulse waveform information.

Vital signs monitors may have a problem under certain circumstances in that since many discrete sensors are used, their attachment to the patient is time-consuming, and the risk that one or more sensors may become unattached is increased. Transport monitoring and emergency room monitoring provide challenges in addition to those normally faced by bedside monitors. Among other issues, the caregivers involved in transport and emergency monitoring have precious little time to attach all of the various sensors to the patient, and to ensure that the sensors remain attached. These problems are exacerbated in tense, unstable situations as may occur at, for example, disaster sites and the battlefield, as well as in non-medical settings as in home care situations.

### BRIEF SUMMARY OF THE INVENTION

What is needed is a small, convenient, and comfortable sensor, as well as a suitable method and system, capable of noninvasively acquiring data useful for measuring blood oxygen saturation, preferably along with one or more additional vital signs such as blood pressure.

One embodiment of the present invention is a noninvasive sensor for use on an anatomical structure of a patient to obtain at least one vital sign, comprising a supportive body; a conformable body coupled to the supportive body and having a contact surface for contacting the anatomical structure; an optical window disposed at the contact surface; and a refraction-mode optical transducer sensitive to arterial oxyhemoglobin saturation, the optical transducer being optically coupled to the optical window. In one exemplary instance of this embodiment, the conformable body comprises a generally disk-shaped body of compressible material, the contact surface being one of the major surfaces of the disk-shaped body; and the optical transducer and the optical window are integrated into a unitary device that is mounted in the compressible material. In another exemplary instance, the noninvasive sensor further comprises a pressure-transmissive medium having a surface disposed at the contact surface; and a pressure transducer coupled to the pressure-transmissive medium for sensing pressure therein.

Another embodiment of the present invention is a noninvasive sensor for use on an anatomical structure of a patient to obtain at least one vital sign, comprising a generally disk-like supportive body; a generally disk-like conformable body coupled to the supportive body and having a contact surface for contacting the anatomical structure; an optical window disposed at the contact surface; a refraction-mode optical transducer sensitive to arterial oxyhemoglobin saturation, the optical transducer being optically coupled to the optical window; and a pressure transducer coupled to the pressure-transmissive medium. The conformable body comprises a generally conformable pressure-transmissive medium comprising a fluid-filled pouch having a surface disposed at the contact surface; a generally annular conformable body having a first surface coupled to the supportive body, and a second surface opposite the first surface, the annular conformable body generally encircling the conformable pressure-transmissive medium; and a generally annular compressive body comprising pressure-attenuating material, the annular compressive body having a first surface abutting the second surface of the annular conformable body, and a second surface disposed at the contact surface, the annular compressive body generally encircling the conformable pressure-transmissive medium.

Another embodiment of the present invention is a system for use on an anatomical structure of a patient to noninvasively obtain at least one vital sign, comprising a generally rigid body; a hold-down assembly incorporated into the body; a retainer extending from the rigid body for engaging the anatomical structure upon activation by the hold-down assembly; and a noninvasive sensor pivotally extending from the body. The noninvasive sensor comprises a supportive body; a conformable body coupled to the supportive body and having a contact surface for contacting the anatomical structure; an optical window disposed at the contact surface; and a refraction-mode optical transducer sensitive to arterial oxyhemoglobin saturation, the optical transducer being optically coupled to the optical window. In one exemplary instance of this embodiment, the noninvasive sensor further comprises a pressure-transmissive medium having a surface disposed at the contact surface; and a pressure transducer coupled to the pressure-transmissive medium for sensing pressure therein.

### BRIEF DESCRIPTION OF THE SEVERAL VIEWS OF THE DRAWINGS

FIG. 1 is a functional block diagram of a sensor-based system for non-invasively monitoring blood pressure and blood oxygen saturation.

FIG. 2 is a schematic drawing of a version of the system of FIG. 1 that is a transportable vital signs monitor in which the sensor assembly and the control and display system are separate and distinct units connected by a cable.

FIG. 3 is a side schematic view of a wrist-mounted sensor assembly that is suitable for the vital signs monitor of FIG. 2.

FIG. 4 is a side schematic drawing of a version of the system of FIG. 1 that is a lightweight self-contained vital signs monitor for mounting on the wrist of a patient, in which the sensor assembly and the control and display system are combined within a common housing.

FIG. 5 is a cross-sectional schematic view of one version of a combined pressure and SpO₂ sensor.

FIG. 6 is a bottom view of the sensor of FIG. 5.

FIG. 7 is a cross-sectional schematic view of another version of a combined pressure and SpO₂ sensor.

FIG. 8 is a bottom view of the sensor of FIG. 7.

FIG. 9 is a cross-sectional schematic view of another version of a combined pressure and SpO₂ sensor.

FIG. 10 is a bottom view of the sensor of FIG. 9.

FIG. 11 is a cross-sectional-schematic view of another version of a combined pressure and SpO₂ sensor.

FIG. 12 is a bottom view of the sensor of FIG. 11.

FIG. 13 is a cross-sectional schematic view of another version of a combined pressure and SpO₂ sensor.

FIG. 14 is a bottom view of the sensor of FIG. 13.

FIG. 15A is a top view of a base section of a sensor suitable for use in the sensor assembly shown in FIG. 3 and in the monitor shown in FIG. 4.

FIG. 15B is a sectional view of the base section of FIG. 15A.

FIG. 15C is a bottom view of the base section of FIG. 15A.

FIG. 16A is a top view of a sensing section of a sensor suitable for use in the sensor assembly shown in FIG. 3 and in the monitor shown in FIG. 4.

FIG. 16B is a sectional view of the sensing section of FIG. 16A.

FIG. 16C is a bottom view of the sensing section of FIG. 16A.

FIG. 17 is a top exploded view of the base section and the sensing section shown in FIGS. 15A-15C and in FIGS. 16A-16C.

FIG. 18 is a bottom exploded view of the base section and the sensing section shown in FIGS. 15A-15C and in FIGS. 16A-16C.

FIG. 19 is a flowchart of an illustrative tissue perfusion method based on spatially distributed SpO₂ measurements.

FIG. 20 is a flowchart of an illustrative tissue perfusion method based on SpO₂ measurements distributed over various pressures during one or more hold down cycles.

FIG. 21 is a cross-sectional schematic view of an SpO₂ sensor having a conformable fluid-filled pouch mounted on a rigid frame, and one or more transducers suitable for SpO₂ measurements mounted to the rigid frame.

FIG. 22 is a cross-sectional schematic view of an SpO₂ sensor having a conformable fluid-filled pouch mounted on a rigid frame, and one or more transducers suitable for SpO₂ measurements mounted to a diaphragm at the bottom of the pouch.

FIG. 23 is a cross-sectional schematic view of an SpO₂ sensor having a compressible body mounted on a rigid frame, and one or more transducers suitable for SpO₂ measurements mounted on the rigid frame.

FIG. 24 is a cross-sectional schematic view of an SpO₂ sensor having a compressible body such as foam mounted on a rigid frame, and one or more transducers suitable for SpO₂ measurements mounted in the compressible body.

### DETAILED DESCRIPTION OF THE INVENTION, INCLUDING THE BEST MODE

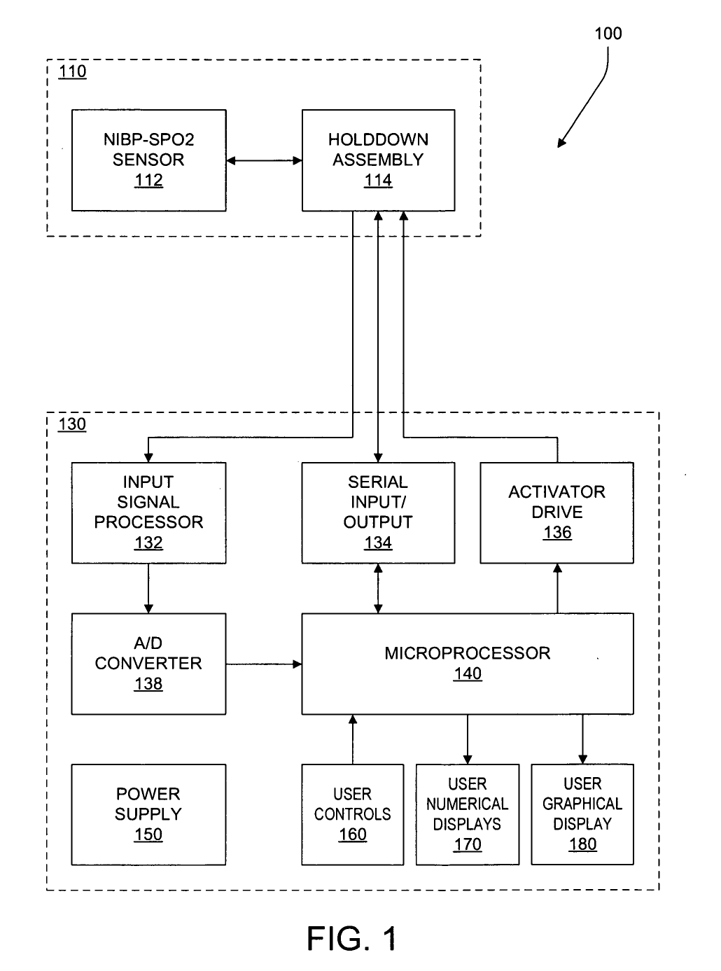

FIG. 1 is a schematic block diagram of an illustrative system 100 suitable for non-invasively monitoring blood pressure “NIBP”) and blood oxygen saturation (SpO₂). Advantageously, a sensor assembly 110 includes a sensor 112 which is capable of providing data for calculating pulse rate as well as both blood pressure (for example, systolic, diastolic, and/or mean pressure) and blood oxygen saturation, and in some embodiments, for calculating perfusion as well. Additionally, other sensors may be included, with the inclusion of a temperature sensor unit 140 being desirable for a vital signs monitor and the inclusion of various additional types of sensors being desirable for a bedside monitor.

The system 100 includes the sensor assembly 110 and a control and display system 130. The sensor assembly 110 and the control and display system 130 may be combined within a common housing, or may be provided as separate and distinct units connected by a cable (not shown). Suitable types of monitoring systems range from lightweight and transportable vital signs monitors to fixed bedside monitors. The sensor assembly 110 includes sensor 112 and holddown assembly 114. The control and display system 130 includes a microprocessor 140, although a controller, logic circuit, or any other type of system suitable for control and display may be used as well. Input signal processor 132 and analog-to-digital converter 138 furnish digitized main and reference channel signals relating to blood pressure and various digitized light intensity signals relating to blood oxygen saturation to a suitable input or inputs of the microprocessor 140. Control and status signals between the sensor assembly 110 and the microprocessor 140 pass through serial input/output circuit 134. An actuator (not shown) in the holddown assembly 114 is controlled by the microprocessor 140 through an actuator drive circuit 136. User control of the microprocessor 140 is done through various controls 160, which vary depending on the type of system, but which may include a keyboard, switches, soft switches, selector knobs, and so forth by which the system may be tested, calibrated, and operated in various modes. Information is displayed to the user through various numerical displays 170 and/or a graphical display 180. A power supply 150 is also provided.

The microprocessor 140 along with associated memory (not shown) controls the actuator drive 136 to vary an applied holddown pressure, and calculates systolic, diastolic, and mean pressure, pulse, blood oxygen saturation, and optionally perfusion from the data from the sensor 112. Additionally, the microprocessor 140 may run various algorithms to compensate for the adverse effects of various actions on the data from the sensor 112, including algorithms to provide high motion tolerance for the blood pressure measurement and to provide noise reduction for the blood oxygen saturation measurement.

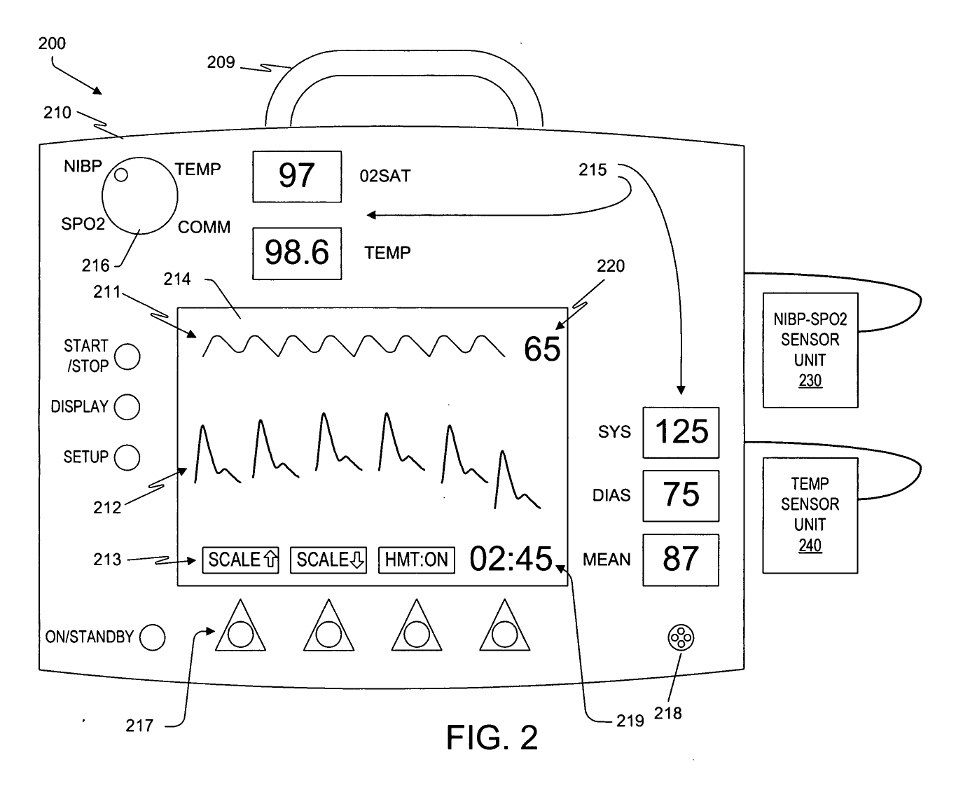

FIG. 2 is a schematic drawing of a version of the system of FIG. 1 that is a vital signs monitor 200 in which a control and display system 110 is separate and distinct from various sensor assemblies, to which it is connected by one or more cables. A suitable transportable universal vital signs monitor is described in U.S. patent application Ser. No. 11/138,953 filed May 26, 2005 (Evans, Universal Transportable Vital Signs Monitor), which hereby is incorporated herein in its entirety by reference thereto. Advantageously, the monitor 210 uses a single sensor unit 230 for both INBP and SpO₂, rather than separate NIBP and SpO₂ sensor units. While a temperature sensor unit 250 is provided as a separate sensor, it may be integrated into the single sensor unit 230 if desired.

The control and display system 210 illustratively has a graphical display 214 that visually displays various waveforms and other information of use to the user. Shown are an SpO₂ waveform 211 and a waveform trend display 212, which shows the patient's arterial waveform in mmHg and is designed for routine monitoring. The graphical display may also display other information as desired, including programmable labels 213 such as “Scale Up,” “Scale Down,” and “HMT:OFF,” which are respectively associated with “soft keys” 217 and which may change with the various display modes of the control and display system 210. The graphical display may also display alphanumeric information, such as the elapsed time 219 of the current measurement period and patient pulse rate 220. Illustratively, various alphanumeric displays 215 are included for displaying alphanumeric information such as the blood oxygen saturation value O2SAT, systolic arterial pressure SYS, diastolic arterial pressure DIAS, mean arterial pressure MEAN, and body temperature TEMP. Illustratively, the alphanumeric displays 215 may include light emitting diodes “LED”). A rotary dial 216 is provided to switch among setup screens for blood oxygen saturation SpO₂, temperature TEMP, non-invasive blood pressure NIBP, and communications COMM. Various hard keys are provided for start/stop, display, setup, and on/standby.

During normal operation, the control and display system 210 displays the SpO₂ waveform 211, the elapsed time 219, the pulse rate 220, and the alphanumeric displays 215 essentially in real time. The waveform trend display 212 may not be in real time where, as in the technique used in the Vasotrac monitor, each waveform is constructed over a period of about 15 seconds based on multiple sensed pressure waveforms over that period. However, the control and display system 210 may be operated in a Real Time Display Mode where the pressure signal as produced by the sweeping action of the sensor unit is displayed. While this mode does show usable arterial waveform information, the scale is not the patient's blood pressure in mmHg. However, the mode may be correlated with the SpO₂ waveform 211.

The control and display system 210 has a plurality of connectors (not shown) which are configured to accept connections to various sensors, including the sensor unit 230 for sensing data related to both non-invasive blood pressure “NIBP”) and blood oxygen saturation (SpO₂). Additionally, other sensors may be included as well, with the inclusion of a temperature sensor unit 140 being desirable for a vital signs monitor and the inclusion of many additional types of sensors being desirable for a bedside monitor.

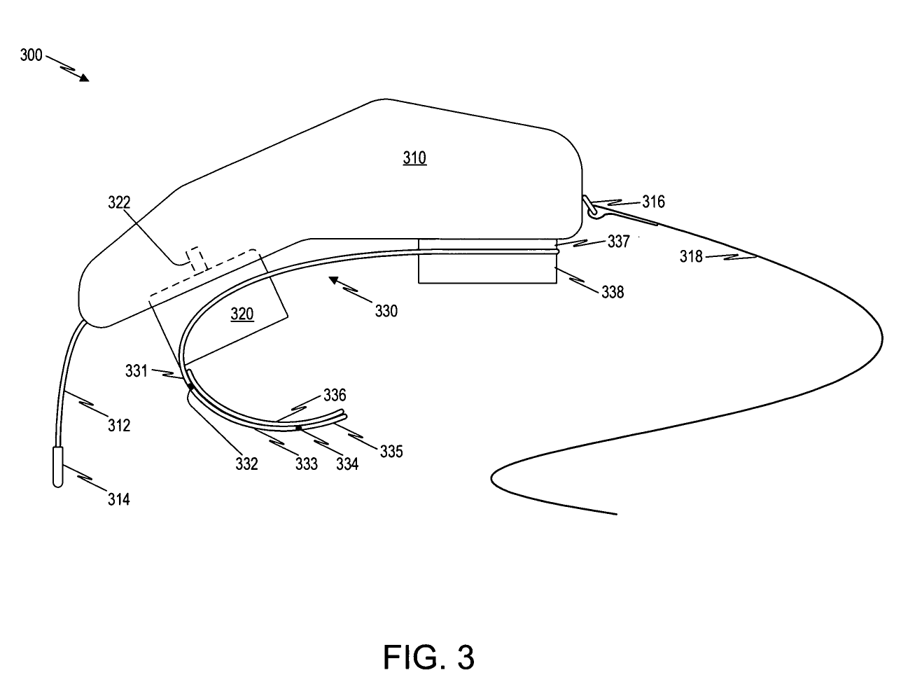

FIG. 3 is a side view of a sensor unit 300 that is suitable for use in the vital signs monitor of FIG. 2. A housing 310 contains a hold-down assembly (not shown) which includes a pair of generally parallel bale cords 312 and a bale 314. A sensor 320 is pivotally connected to the hold-down assembly by a pivot rod 322. An articulated placement guide 330 having sections 331, 333 and 335 backed by flexible layer 336 with intervening spaces 332 and 334 is used to properly position and stabilize the sensor 320 on the wrist of a patient. The placement guide 330 is attached at one end of the section 331 to the casing 310 by the mounting block 337. An illustrative articulated placement guide is described in further detail in U.S. patent application Ser. No. 11/072,199 filed Mar. 4, 2005 (Kevin R. Evans, “Articulated placement guide for sensor-based noninvasive blood pressure monitor”), which hereby is incorporated herein in its entirety by reference thereto. However, other types of placement guides including fixed size guides may be used as well, if desired.

The sensor unit 300 is secured to the patient in any convenient manner, illustratively by strapping it on with a Velcro® brand strap 318. The ends of the strap 318 are looped through bale 314 and anchor 316, which are attached at or near opposite ends of the sensor unit 300. The anchor 316 is illustratively a U-shaped metal bracket that rotatably projects from the casing 310. The bale 314 is a slotted plastic body which is molded about the pair of bale cords 312, and receives the end of the strap 318. When the sensor unit 300 is applied to the patient, the placement guide 330 straddles the styloid process bone of the patient and generally guides the sensor 320 into position over the underlying artery and the radius bone. Proper placement may be verified tactilely by passing a finger between the bail cords 312 and an access notch in the placement guide segments 331 and 333, and feeling the distal edge of the radius bone. The access notch extends from a generally circular aperture through which the sensor 320 moves.

Blood pressure and blood oxygen saturation may be determined from measurements made non-invasively by the sensor unit 300 at the surface of a patient's body in the following manner. A user positions the sensor 300 over an artery of the patient on a suitable location such as, for example, on the wrist over the edge of the radius bone, using the placement guide 330. At the initiation of a monitoring cycle, a varying force is applied to the radial artery and the counter pressure is sensed by the sensor 320. This counter pressure includes pressure pulses from the radial artery, and is digitized and used to calculate blood pressure. Additionally, an optical signal indicative of the blood oxygen saturation of the blood under the sensor 320 is sensed, and is digitized and used to calculate blood oxygen saturation. Measurements may be made over one or more cycles, to perform spot monitoring or continuous monitoring. As the hold-down assembly operates, it draws in the bale 314 via the bale cords 312, so that sensor 320 gently exerts pressure against the patient's wrist over the radial artery, while cushion 338 on the placement guide segment 331 and layer 336 extending across whole or parts of placement guide segments 331, 333 and 335, and spanning intervening gaps 332 and 334, gently distribute pressure over other areas of the patient's wrist. The cushion 338 also functions as a pivot point about which the hold-down pressure is applied, while the layer 336 also enables articulation. While an articulated guide 330 is advantageous to achieve a universal device, non-articulated guides may be used instead, if desired.

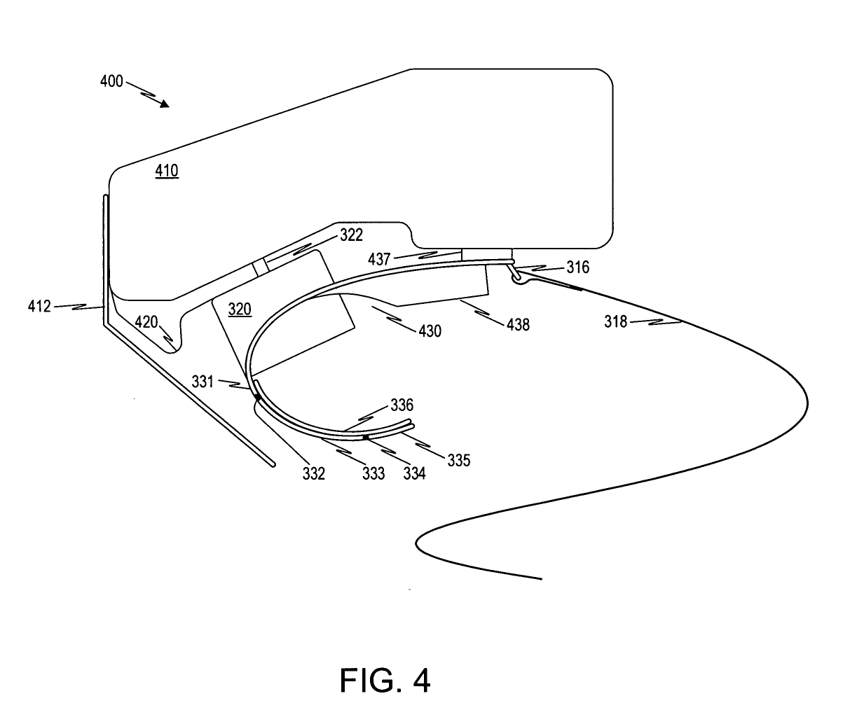

FIG. 4 is a side schematic drawing of a lightweight self-contained vital signs monitor for mounting on the wrist of a patient, in which the sensor assembly and the control and display system are combined within a common housing. An example of a suitable monitor is described in U.S. patent application Ser. No.11/072,916 filed Mar. 4, 2005 (Evans, Sensor-Based Apparatus and Method for Portable Noninvasive Monitoring of Blood Pressure, Attorney Docket No. 01845.0042-US-01), which hereby is incorporated herein in its entirety by reference thereto. The monitor 400 has a housing 410 which contains a hold-down pressure generating unit (not shown) mounted therein. The sensor 320 is pivotally coupled to the hold-down pressure generating unit by the pivot rod 322. The hold-down pressure generating unit illustratively includes a control circuit (not shown), a pneumatic system (not shown), and a power source. A user interface panel mounted on the face of the housing 410 is electrically coupled to the control circuit, and includes a numeric indicators for displaying systolic pressure, diastolic pressure, pulse or heart rate, and SpO₂. The user interface panel also includes a start/stop switch (not shown), which is pressed to initiate a monitoring cycle similar to that described above. Various other indicators and controls may be included if desired. An electrical connector 52, illustratively a ribbon cable, may be used to electrically connect the sensor 320 to the control circuit in the housing 410. An illustrative placement guide 430 is very similar to the placement guide 330 of FIG. 3, except that the mounting block 437 and the cushion 438 are slightly different than the corresponding mounting block 337 and cushion 338 of FIG. 3. The techniques of attaching the monitor 400 to the patient and performing measurements is substantially the same as described for the system 200 (FIG. 2), except that the monitor 400 is entirely self-contained.

Other noninvasive sensor-based monitors for monitoring blood pressure, including systolic pressure, diastolic pressure, and pulse rate, may be modified in accordance with the principles described herein for performing SpO₂ measurements. Some examples are described in U.S. Pat. No. 5,797,850 issued Aug. 25, 1998 to Archibald et al., U.S. Pat. No. 5,640,964 issued Jun. 24, 1997 to Archibald et al., and U.S. Pat. No. 6,558,335 issued May 6, 2003, to Thede.

Advantageously, the sensor 320 is relatively small compared to such devices as cuffs used with the oscillometric and auscultatory methods, the sensor 320 applies a hold down pressure to only a relatively small area above the underlying artery of the patient. Consequently, blood pressure and blood oxygen saturation measurements may be taken with less discomfort to the patient. Because the sensor 320 does not require inflation or deflation, faster and more frequent measurements may be taken. Furthermore, the sensor 320 better conforms to the anatomy of the patient so as to be more comfortable to the patient, and the improved accuracy and repeatability of placement and the automatic application of the hold-down pressure avoids ineffective hold-down cycles and achieves consistent and accurate blood pressure and blood oxygen saturation measurements.

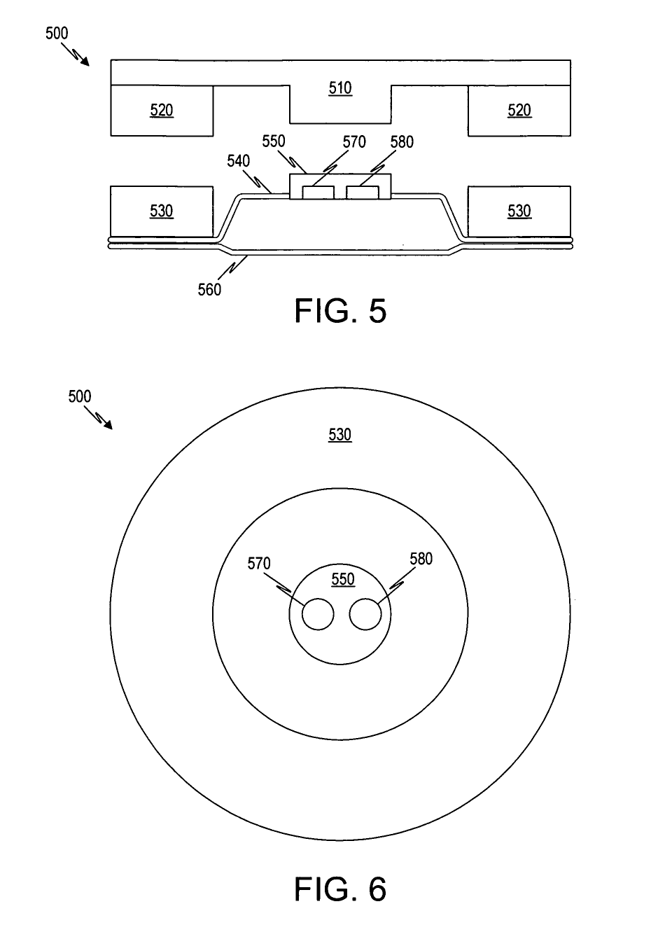

The sensor 320 may be configured in various ways, with illustrative examples being shown in FIGS. 5-14. The portion of the sensor 320 that is operationally in contact with the patient while measurements are being made may be thought of as a contact section. Advantageous features common to the examples of FIGS. 5-14 include a base section which has a rigid frame 510 to which is mounted a conformable ring 520. In the examples of FIGS. 5-14, the contact section is a surface of a generally disc-like fluid-filled pouch generally contained within a compressible ring 530. The pouch is formed by a diaphragm 560 which is operationally in contact with the patient, and a diaphragm 540 which preferably is operationally deformable within the interior of the sensor to help reduce differences in pressure exerted by the pouch and the compressible ring 530. The fluid contained within the pouch preferably is an incompressible liquid for conveying pressure pulses, although other pressure conveying materials such as gels and certain solids may be used if desired. The diaphragms 540 and 560 are bonded to one another along respective peripheries, and the diaphragm 540 is bonded along its periphery to the compressible ring 530. A transducer housing 550 is bonded to the diaphragm 540, and includes a pressure transducer 570 in fluid communication with the incompressible liquid through an orifice in the housing 550. Although illustratively shown as a two piece type with a disposable contact section and a reusable base section, the sensors shown in FIGS. 5-14 may be made as a reusable one-piece sensor. For the two-piece type, the housing 550 also functions as a mechanical and electrical connector that mates with a corresponding connector of any suitable type on the reusable base section, illustratively represented by a projecting region of the frame 510. For the one-piece type, the pressure transducer 570 may be mounted on the frame 510 and the diaphragm 540 may be bonded to the frame 510 such that the pressure transducer 570 is in fluid communication with the incompressible liquid.

The illustrative examples of the sensor 320 shown in FIGS. 5-14 also have in common the use of one or more pulse oximeter transducers of the LED-type. In each example, the pulse oximeter transducers illuminate the patient through an optical window in the contact surface, which is non-opaque and preferably transparent at the wavelength of the pulse oximeter transducer. While the optical window preferably is part of a clear flexible plastic sheet, it may be any other non-opaque material and may even be an opening in a contact surface.

FIGS. 5 and 6 show a version 500 of the sensor which includes an LED-type SpO₂ transducer 580 for purposes of pulse oximetry. Pulse oximetry provides estimates of arterial oxyhemoglobin saturation (SaO₂) by utilizing selected wavelengths of light to noninvasively determine the saturation of oxyhemoglobin (SpO₂). The transducer 580 is positioned to illuminate the surface of a patient's skin through the fluid-filled pouch, and particularly through diaphragm 560 and the incompressible fluid. The diaphragm 560 and the incompressible fluid are selected so as not to excessively attenuate the wavelengths used for the measurement.

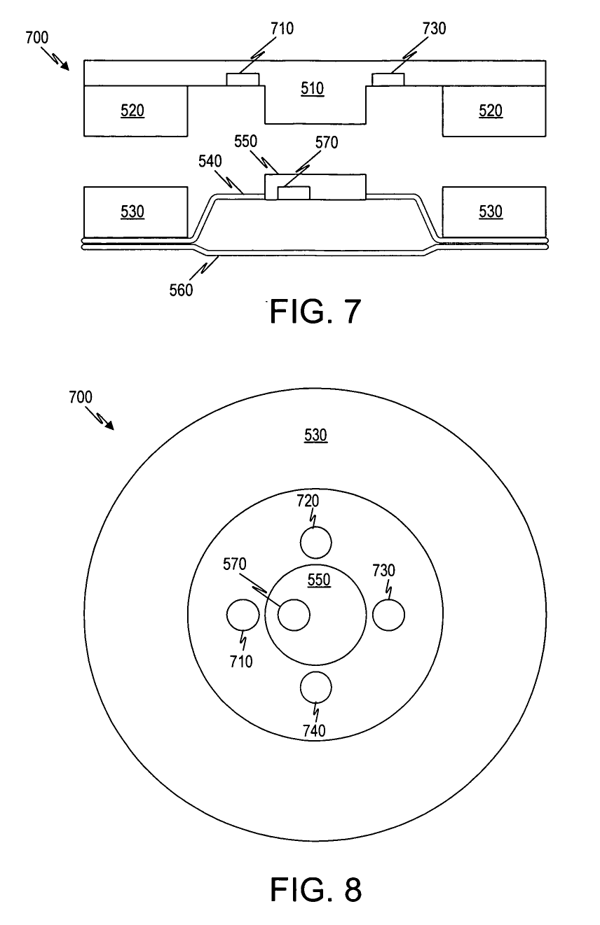

FIGS. 7 and 8 show a version 700 of the sensor which includes multiple LED-type SpO₂ transducers, illustratively four transducers 710, 720, 730 and 740, which are mounted on the frame 510 in a circular pattern and are spaced away from the edge of the housing 550. The transducers 710, 720, 730 and 740 are positioned to illuminate the surface of a patient's skin through the fluid-filled pouch, and particularly through diaphragms 540 and 560 and the incompressible fluid therebetween. The diaphragms 540 and 560 and the incompressible fluid therebetween are selected so as not to excessively attenuate the wavelengths used for the measurement. The use of an array of SpO₂ transducers provides multiple measurements, which may be used to determine perfusion as well as SpO₂.

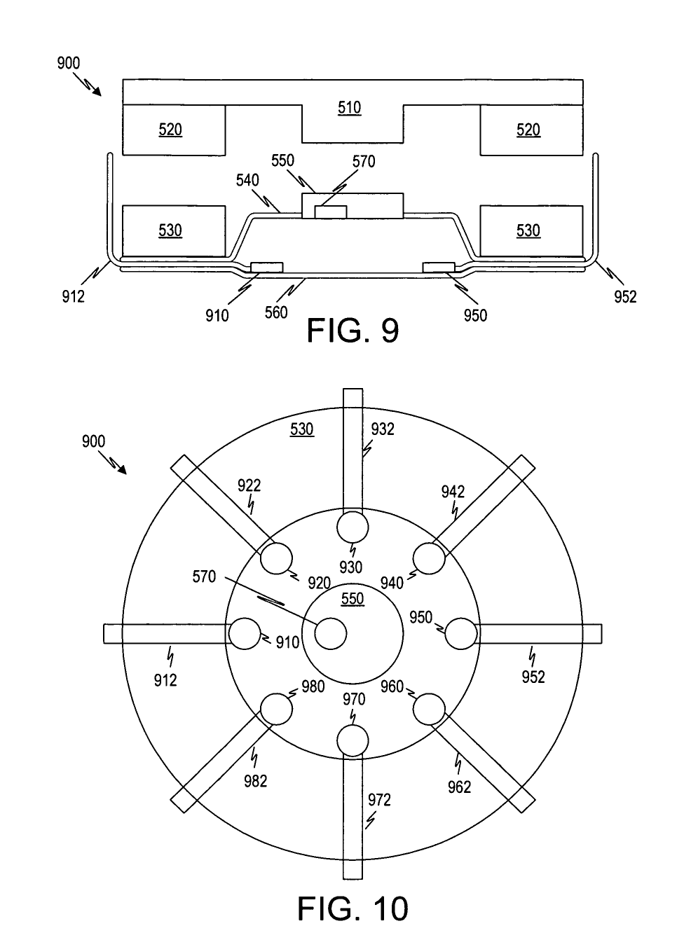

FIGS. 9 and 10 show a version 900 of the sensor which has an array that is similar to that of the sensor 700 (FIGS. 7 and 8) but which uses multiple LED-type SpO₂ transducers mounted on, illustratively, the diaphragm 560 within the fluid filled pouch. Illustratively eight transducers 910, 920, 930, 940, 950, 960, 970 and 980 are mounted on the diaphragm 560, and respective leads 912, 922, 932, 942, 952, 962, 972 and 982 extend from the transducers and back into the sensor frame 510 (not shown). The use of an array of SpO₂ transducers provides multiple measurements which may be used to determine perfusion as well as SpO₂.

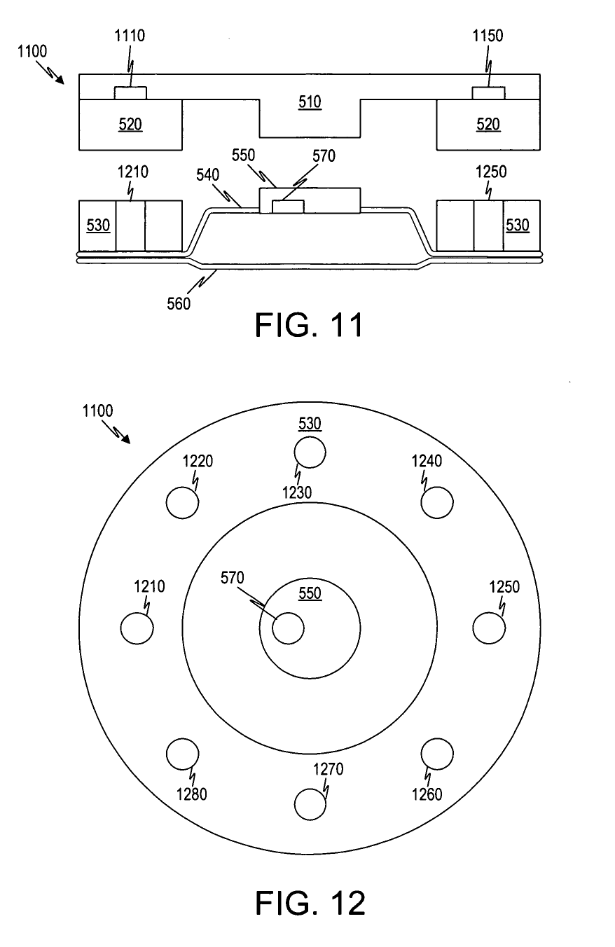

FIGS. 11 and 12 show a version 1100 of the sensor which includes multiple LED-type SpO₂ transducers, illustratively eight transducers 1110, 1120, 1130, 1140, 1150, 1160, 1170 and 1180, which are mounted on the frame 510 in a circular pattern over the conformable ring 520 and over respective optically non-opaque and preferably transparent channels 1210, 1220, 1230, 1240, 1250, 1260, 1270 and 1280 in the compressible ring 530. The fluid and walls of the conformable ring 520 along with the channels 1210, 1220, 1230, 1240, 1250, 1260, 1270 and 1280 are selected so as not to excessively attenuate the wavelengths used for the measurement. The use of a large array of SpO₂ transducers provides multiple measurements over a greater area than the second embodiment, which may be used to determine perfusion as well as SpO₂.

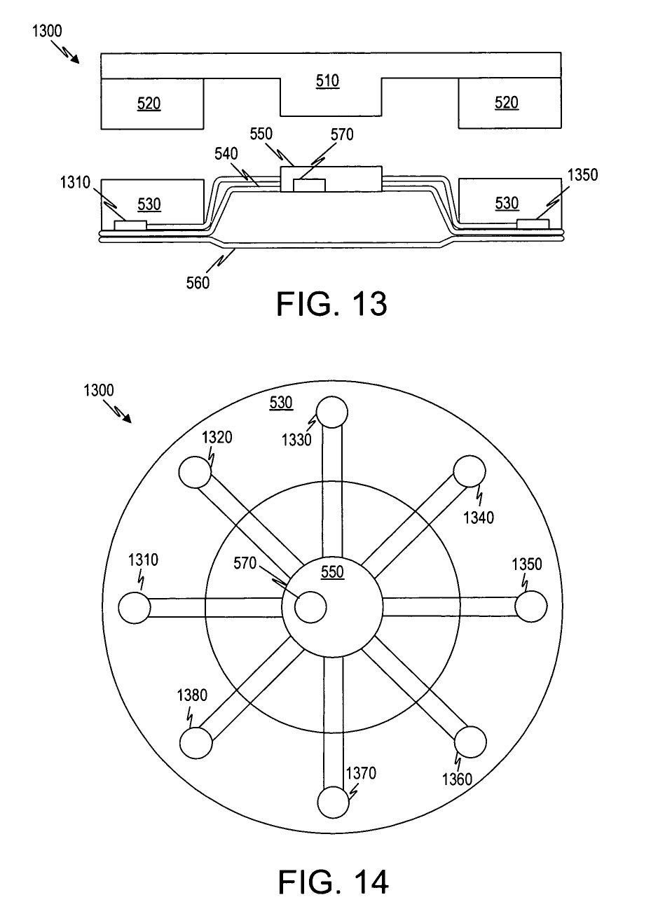

FIGS. 13 and 14 show a version 1300 of the sensor which has an array that is similar to that of the sensor 1100 (FIGS. 11 and 12) but which uses multiple LED-type SpO₂ transducers mounted directly into the compressible pad 530. Illustratively eight transducers 1310, 1320, 1330, 1340, 1350, 1360, 1370 and 1380 are embedded into the compressible ring 530, and respective leads extend from the transducers to the transducer housing 550. Alternatively, the transducers 1310, 1320, 1330, 1340, 1350, 1360, 1370 and 1380 may be recessed into the compressible ring 530, and optically non-opaque and preferably transparent channels (not shown; see FIG. 11) may be provided between the transducers 1310, 1320, 1330, 1340, 1350, 1360, 1370 and 1380 and the diaphragm 540. The use of an array of SpO₂ transducers provides multiple measurements over a greater area than the second embodiment, which may be used to determine perfusion as well as SpO₂.

While various suitable types of transducers suitable for blood oxygen saturation measurements are well known and smaller and more effective versions will become available, one suitable type of transducer is the type LNOPv® sensor available from the Masimo Corporation of Irvine, Calif. Suitable interface circuitry for the various types of SpO₂ transducers are also well known, and include the MS board for the LNOPy sensor, which is also available from the Masimo Corporation.

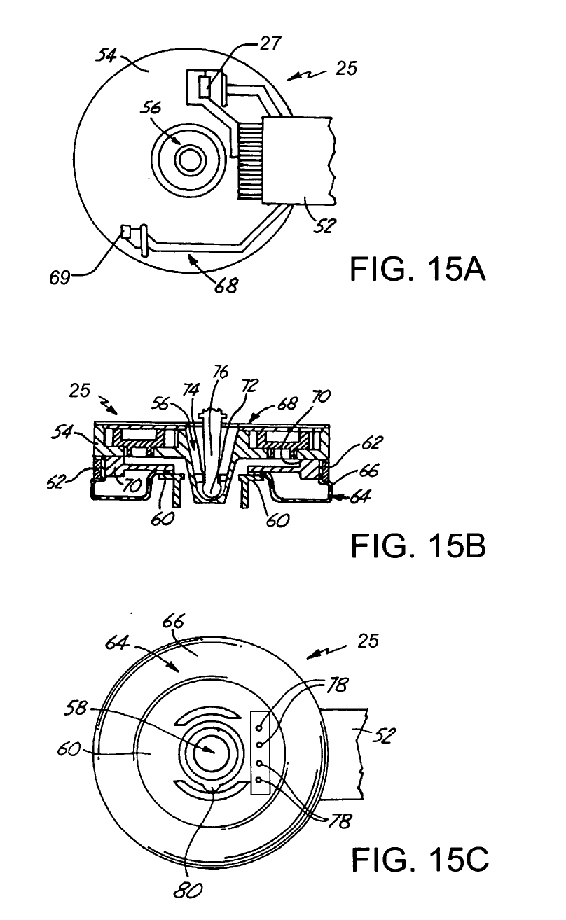

Additional technical aspects of the sensor 320 are shown in the illustrative sensor detail of FIGS. 15A-15C, 16A-16C, 17 and 18. The SpO₂ transducers and associated leads may be positioned in various ways as illustratively shown in FIGS. 5-14, and are omitted from FIGS. 15A-15C, 16A-16C, 17 and 18 for clarity. The sensor shown in these figures is illustratively a two-part sensor design in which the part of the sensor that contacts the patient is replaceable.

FIGS. 15A, 15B and 15C show top, sectional, and bottom views, respectively, of a base section 25 of the two-part sensor. Base section 25 includes a top plate 54, an upper receptacle 56, a lower receptacle 58, an inner mounting ring 60, an outer mounting ring 62, and a flexible ring 64. The flexible ring 64 is defined by side wall diaphragm 66 and upper capture 70. The outer edge portion of diaphragm 66 is held between top plate 54, outer ring 62 and upper capture 70, while the inner edge portion of diaphragm 66 is held between inner ring 60 and upper capture 70. The flexible ring 64 is filled with fluid, and is deformable in the vertical direction so as to be able to conform to the contour of the anatomy of the patient surrounding the underlying artery. Because fluid is permitted to flow through and around ring 64, pressure is equalized around the patient's anatomy.

The base section 25 also includes a pivot mount 72 for pivotally joining the sensor to a pivot post (not shown) that extends from the hold-down assembly. The pivot mount 72 allows the sensor to pivot near the wrist surface to accommodate a range of patient anatomies.

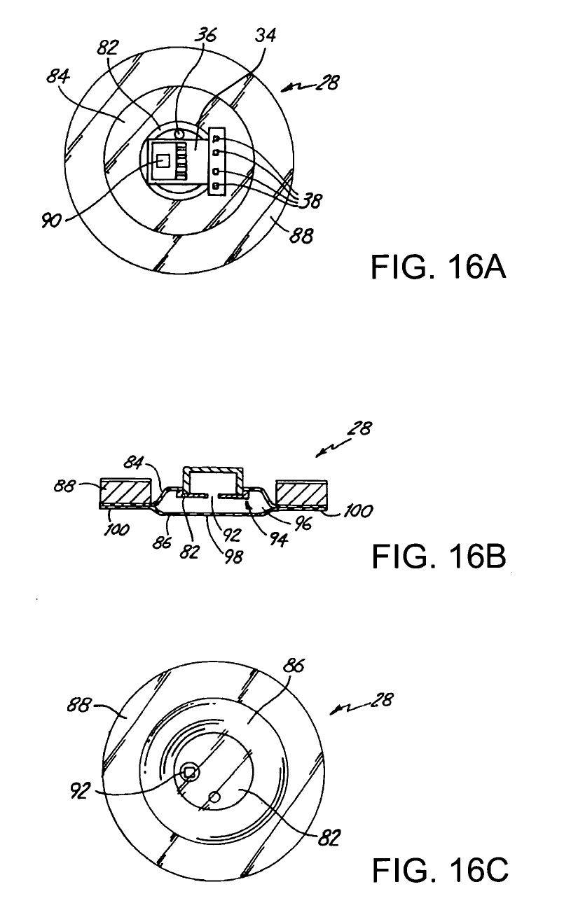

The base section 25 receives a sensing section 28 (see FIGS. 16A-16C), and includes electrical connectors 78 and an alignment receptacle 80 in, illustratively, the inner mounting ring 60 of the lower receptacle 58, for receiving a mating connector 34 (see FIGS. 16A & 17) in the sensing section 28. The sensing section 28 may be permanently joined or detachably joined to the base section 25.

The base section 25 also includes a reference channel pressure transducer 27, an electrical circuit 68 that includes a memory chip 69, and an electrical connector 52, illustratively a ribbon cable, for power and communication of pressure signals from transducers 27 and 90 (FIG. 15A) in the sensor and for communication of data to and from the memory chip 69. Power and communication with the transducer 90 is through the connectors 78.

FIGS. 16A-16C show top view, sectional and bottom views, respectively, of sensing section 28 of the sensor. Sensing section 28 includes a diaphragm capture 82, an inner diaphragm 84, a flexible (or outer) diaphragm 86, a compressible ring 88, a main channel pressure transducer 90 having a sensing surface 92, and connector 34. Inner diaphragm 84 and flexible diaphragm 86 form a sensor chamber 94 which is filled with preferably a fluid coupling medium 96.

Any of a variety of different types of pressure transducers may be used for the main channel transducer 90 and the reference channel transducer 27, one suitable type being part number MPX2300DT1 or MPX2301DT1, which is available from Freescale Semiconductor, Inc. of Austin, Tex., and from Motorola Inc. of Tempe, Ariz.

The connector 34 illustratively includes an alignment element 36 and electrical connectors 38. Electrical connectors 38 are connected to and extend from pressure transducer 90. Electrical connectors 38 mate with electrical connectors 78 located on the base section 26. Electrical connectors 38 provide the connection between transducer 90 and the electrical circuitry of the base section 26. Alignment element 36 is received by alignment receptacle 80 (FIG. 8C) of base section 25 to precisely position electrical connectors 38 within the corresponding electrical connectors 78 of the base section 25. It will be appreciated that any suitable mating electrical connectors may be used for the electrical connectors 38 and 78; illustratively, electrical connectors 38 are receptacles or sockets, while electrical connectors 78 are recessed pins.

Compressible ring 88 is generally annular and may be formed from a polyurethane foam or other compressible material that also has pressure pulse dampening properties, including open cell foam and closed cell foam. Ring 88 is centered about flexible diaphragm 86 and positioned above diaphragms 84 and 86. Compressible ring 88 is isolated from the fluid coupling medium 96 within sensor chamber 94. The compressibility of ring 88 allows ring 88 to absorb and dampen forces in a direction parallel to the underlying artery. These forces are exerted by the blood pressure pulses on sensing section 28 as the blood pressure pulses cross flexible diaphragm 86. Because compressible ring 88 is reasonably well isolated from fluid coupling medium 96, the forces absorbed or received by ring 88 are not well transmitted to fluid coupling medium 96. Instead, these forces are transmitted across compressible ring 88 and flexible ring 64 to top plate 54 (shown in FIG. 15B), which is a path distinct and separate from fluid coupling medium 96.

Rings 64 and 88 apply force to the anatomy of the patient to neutralize the forces exerted by tissue surrounding the underlying artery. Rings 64 and 88 are compressible in height, thus the height of the side of the sensor 20 decreases as the sensor 20 is pressed against the patient's wrist.

Inner diaphragm 84 is an annular sheet of flexible material having an inner diameter sized to fit around diaphragm capture 82. An inner portion of inner diaphragm 84 is trapped or captured, and may be adhesively affixed to the lip of diaphragm capture 82. Inner diaphragm 84 is permitted to initially move upward as flexible diaphragm 86 conforms to the anatomy of the patient surrounding the underlying artery. As compressible ring 88 is pressed against the anatomy of the patient surrounding the artery to neutralize or offset forces exerted by the tissue, flexible diaphragm 86 is also pressed against the anatomy and the artery. However, because inner diaphragm 84 is permitted to roll upward, sensor chamber 94 does not experience a large volume decrease or a large corresponding pressure increase. Thus, greater force is applied to the anatomy of the patient through compressible ring 88 to neutralize tissue surrounding the artery without causing a corresponding large, error-producing change in pressure within sensor chamber 94 as the height of the side wall changes and the shape of flexible diaphragm 86 changes. As a result, the sensor 20 achieves more consistent and accurate blood pressure measurements.

Flexible diaphragm 86 is a generally circular sheet of flexible material capable of transmitting forces from an outer surface to fluid coupling medium 96 within sensor chamber 94. Diaphragm 86 is coupled to inner diaphragm 84 and is configured for being positioned over the anatomy of the patient above the underlying artery. Diaphragm 86 includes an active portion 98 and a nonactive portion 100 or skirt. Non-active portion 100 constitutes the area of diaphragm 86 where inner diaphragm 84 is heat sealed or bonded to diaphragm 86 adjacent compressible ring 88. Active portion 98 of flexible diaphragm 86 is not bonded to inner diaphragm 84, and is positioned below and within the inner diameter of ring 88. Active portion 98 of diaphragm 86 is the active area of sensing section 28 which receives and transmits pulse pressure to pressure transducer 90.

Fluid coupling medium 96 within sensor chamber 94 may be any fluid (gas or liquid) capable of transmitting pressure from flexible diaphragm 86 to transducer 90. Alternatively, another pressure pulse transmission medium may be used, including a medium made of a solid material or materials, or combinations of different materials, solid and fluid. Fluid coupling medium 96 interfaces between active portion 98 of diaphragm 86 and transducer 90 to transmit blood pressure pulses to transducer 90. Because fluid coupling medium 96 is contained within sensor chamber 94, which is isolated from compressible ring 88 of sensing section 28, fluid coupling medium 96 does not transmit blood pressure pulses parallel to the underlying artery, forces from the tissue surrounding the underlying artery, and other forces absorbed by compressible ring 88 to transducer 90. As a result, sensing section 28 more accurately measures and detects arterial blood pressure.

Sensing section 28 permits accurate and consistent calculation of blood pressure. Although blood pressure pulses are transmitted to the transducer 90 through hole 92, sensing section 28 is not dependent upon precisely accurate positioning of the sensor over the underlying artery because of the large sensing surface of the active portion 98 of the flexible diaphragm 86. Thus, the sensor is tolerant to some sensor movement as measurements are being taken.

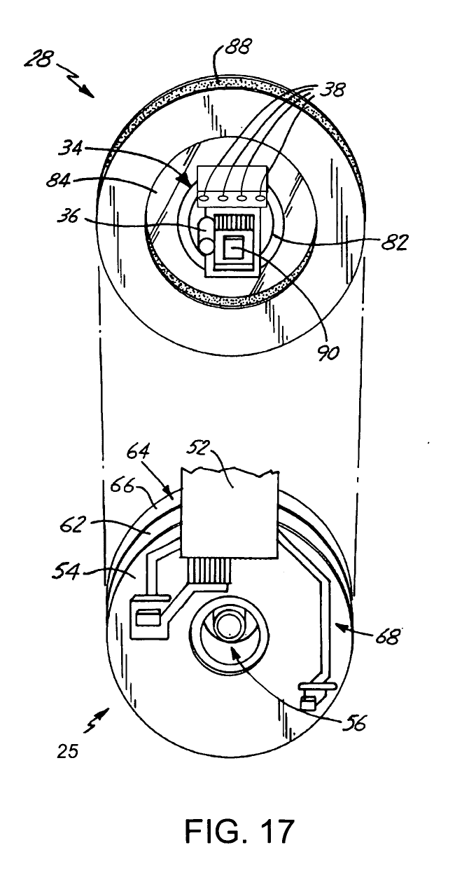

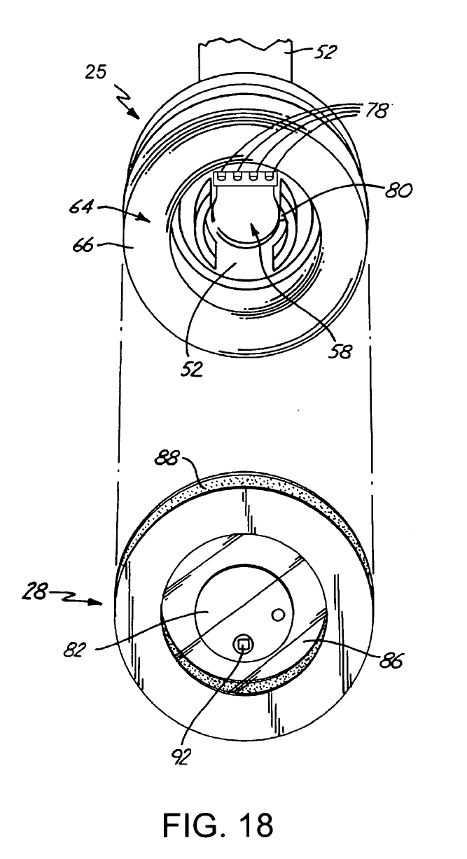

FIG. 17 is a top exploded view of the base section 25 and the sensing section 28 and FIG. 18 is a bottom exploded view of the base section 25 and the sensing section 28. When assembled, flexible ring 64 and compressible ring 88 form the side wall of the sensor 20. The connector 34 of sensing section 28 may be used to detachably connect sensing section 28 to base section 25.

The sensor achieves a zero pressure gradient across active portion 98 of the sensing section 28, achieves a zero pressure gradient between transducer 90 and the underlying artery, attenuates or dampens pressure pulses that are parallel to sensing surface 92 of transducer 90, and neutralizes forces of the tissue surrounding the underlying artery. The sensor contacts and applies force to the anatomy of the patient across non-active portion 100 and active portion 98 of flexible diaphragm 86. However, the pressure within sensor chamber 94 is substantially equal to the pressure applied across active portion 98 of flexible diaphragm 86. In addition, because fluid coupling medium 96 within sensor chamber 94 is isolated from ring 88, pressure pulses parallel to the underlying artery, forces from tissue surrounding the underlying artery, and other forces absorbed by ring 88 are not transmitted through fluid coupling medium 96 to transducer 90. Consequently, the sensor also achieves a zero pressure gradient between transducer 90 and the underlying artery. The remaining force applied by the sensor across non-active portion 100, which neutralizes or offsets forces exerted by the tissue surrounding the underlying artery, is transferred through the side wall (rings 64 and 88) to top plate 54. As a result, the geometry and construction of the sensor provides a suitable ratio of pressures between non-active portion 100 and active portion 98 of flexible diaphragm 86 to neutralize tissue surrounding the underlying artery and to accurately measure the blood pressure of the artery.

If desired, sensing section 28 may be made detachably connected to base section 25 such that sensing section 28 may be replaced if contaminated or damaged, or if it is desired to use a new disposable contact element with each new patient. Although the sensor is described as having a distinct base section 26 and a distinct sensing section 28 which includes the pressure transducer 90, the sensor need not comprise distinct base and sensing sections. Although the sensor is described as a unitary structure in which the pressure transducer 90 is mounted to the sensing section 28, various components of the sensor such as the pressure transducer 90 may be distributed. As an example, the pressure transducer may be mounted to a different structure away from the base, and placed in fluid communication with the sensing surface through a fluid-filled tube.

Various methods for calculating blood pressure are known and available. Some particularly suitable methods for the sensor 320 include the basic algorithm described in U.S. Pat. No. 5,797,850 issued Aug, 25, 1998 to Archibald et al., the beat onset detection method described in U.S. Pat. No. 5,720,292 issued Feb. 24, 1998 to Poliac, the segmentation estimation method described in U.S. Pat. No. 5,738,103 issued Apr. 14, 1998 to Poliac, and the high motion detection algorithm described in U.S. patent application Ser. No. 11/121,305 filed May 2, 2005 (Lunak et al., Noninvasive Blood Pressure Monitor Having Automatic High Motion Tolerance, Attorney Docket No. 01845.0047-US-01), all of which are incorporated herein in their entirety by reference thereto.

Various methods for calculating SpO₂ that are suitable for use with the sensor 320 are known and available. Illustrative methods are well known and are described in many publications, including the following publications: The Nellcor Corporation, Monitoring Oxygen Saturation with Pulse Oximetry, 2003; and Severinghaus, J. W. Simple, Accurate equations for human blood O₂ dissociation computations, J Appl Physiol. 46(3): 599-602, 1979.

Various techniques may be used to position the sensor for purposes of the pulse oximetry measurement. Proper location of the radial artery pulse oximeter transducer may be determined by scanning the pulse oximeter measurements as the sensor is moved over the radial artery region, the proper location of the pulse oximetry transducer being indicated by the maximum measured SpO₂. Where the sensor is also a blood pressure sensor, this technique may be used instead of a placement guide for positioning the sensor for the blood pressure measurement as well as the pulse oximetry measurement. Alternatively where an array of pulse oximeter transducers is used, the sensor may be placed using other techniques such as a placement guide, and the pulse oximeter transducer yielding the maximum measured SpO₂ is selected for the radial artery pulse oximetry measurements.

The SpO₂ sensor arrays described in FIGS. 8 through 14 are also suitable for determining tissue perfusion at any particular time before, after or during a hold down cycle. In one illustrative approach that uses an array of pulse oximeter transducers, one of the pulse oximeter transducers is placed directly over the radial artery using manual placement or array selection as described above, or any other suitable technique. This pulse oximeter transducer is used for an artery blood oxygenation measurement, and one or more of the other pulse oximeter transducers that are sufficiently displaced so as to be measuring capillary blood oxygenation are used for the capillary blood oxygenation measurement. The relative difference between the radial artery and capillary blood oxygenation measurements may be adaptable to a tissue perfusion efficiency coefficient of the following form:
Tissue Perfusion Coefficient=(SaO₂−ScO₂)/SaO₂
wherein SaO₂ is the radial artery blood oxygenation reading in percent, and ScO₂ is the capillary blood oxygenation reading in percent. Calculated in this manner, the tissue perfusion efficiency would be inversely proportional to the tissue perfusion coefficient described above.

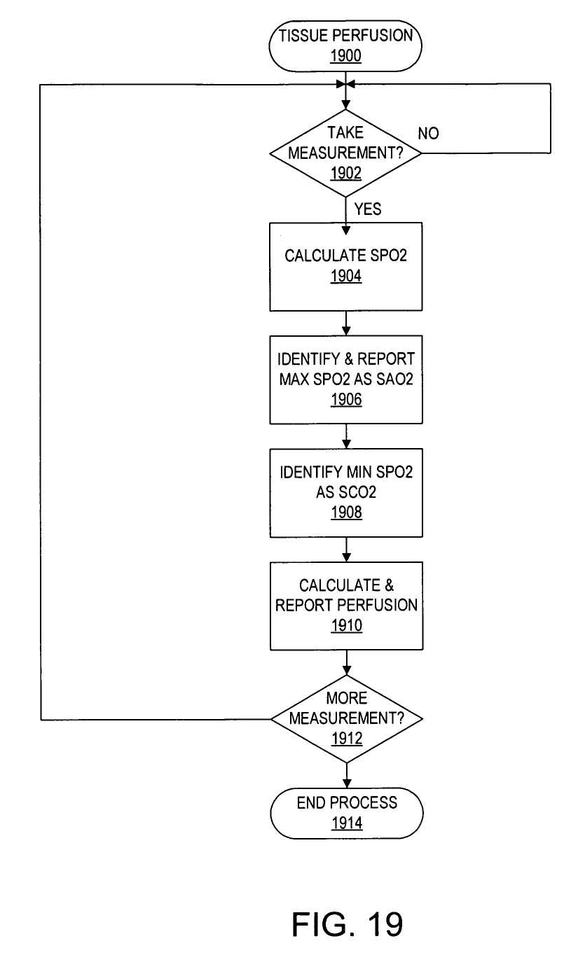

FIG. 19 is a flowchart of an illustrative tissue perfusion method 1900 based on spatially distributed SpO₂ measurements. If the sensor device is ready to take a measurement (block 1902—yes), SpO₂ values are calculated using signals acquired from various pulse oximeter transducers in the array (block 1904). Depending on the algorithm used, one or many signals from the pulse oximeter transducers may be used to calculate SpO₂ values. The pulse oximeter transducers from which signals are used for the calculations may be pre-selected based on previously calculated SpO₂ maximum and minimum values during a positioning cycle or during a previous cycle or group of cycles, or SpO₂ values may be calculated for all of the pulse oximeter transducers and the maximum and minimum values may be selected dynamically based on the SpO₂ values calculated in each cycle or group of cycles. The maximum SpO₂ value is identified and reported as the SaO₂ reading (block 1906), and the minimum SpO₂ value is identified (Block 1908) and used as the ScO₂ value along with the SaO₂ value to calculate and report a perfusion coefficient (block 1910). If more measurements are desired (block 1912—yes), processing resumes from block 1902. Otherwise, processing terminates (block 1914).

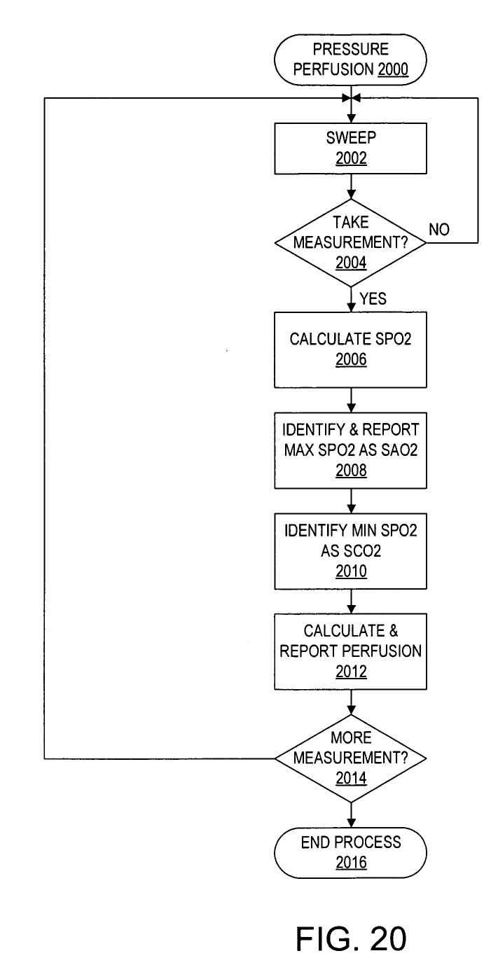

FIG. 20 is a flowchart of an illustrative tissue perfusion method 2000 based on SpO₂ measurements distributed over various pressures during one or more hold down cycles. A hold down cycle such as a sweep cycle is initiated (block 2002). If the sensor device is ready to take a measurement (block 2004—yes), SpO₂ values are calculated using signals acquired from various pulse oximeter transducers in the array (block 2006). Depending on the algorithm used, one or many signals from the pulse oximeter transducers may be used to calculate SpO₂ values during one or more entire hold down cycles, or during portions of one or more hold down cycles. The pulse oximeter transducers from which signals are used for the calculations may be pre-selected based on previously calculated SpO₂ maximum and minimum values during a positioning cycle or during a previous cycle or group of cycles, or SpO₂ values may be calculated for all of the pulse oximeter transducers and the maximum and minimum values may be selected dynamically based on the SpO₂ values calculated in each cycle or group of cycles. The maximum SpO₂ value is identified and reported as the SaO₂ reading (block 2008), and the minimum SpO₂ value is identified (block 2010) and used as the ScO₂ value along with the SaO₂ value to calculate and report a perfusion coefficient (block 2012). If more measurements are desired (block 2014—yes), processing resumes from block 2002. Otherwise, processing terminates (block 2016).

Temperature may be incorporated into the system in various ways. While various suitable types of temperature sensors are and will become available, an illustrative type of temperature sensor is placed in the patient's ear, inasmuch as the sensor is easy to use and the same-sized sensor works for both smaller and larger patients. This type of sensor is typically inserted into a patient's ear, and functions essentially independent of patient weight or size. A suitable model of temperature sensor is the Genius Model 8300G Tympanic Thermometer, which is available from Sherwood Davis & Geck of Watertown, N.Y.

A noninvasive core body temperature transducer may also be incorporated into the sensor 320 by being mounted at or near the surface of the sensor 320 that contacts the patient's skin, or may be located elsewhere on the sensor unit, such as at the surface of the cushion 330 of the sensor unit 300, or the cushion 438 of the sensor unit 400. An example of a noninvasive core body temperature transducer and associated algorithm is disclosed in U.S. Pat. No. 6,827,487, issued Dec. 7, 2004 to Baumbach.

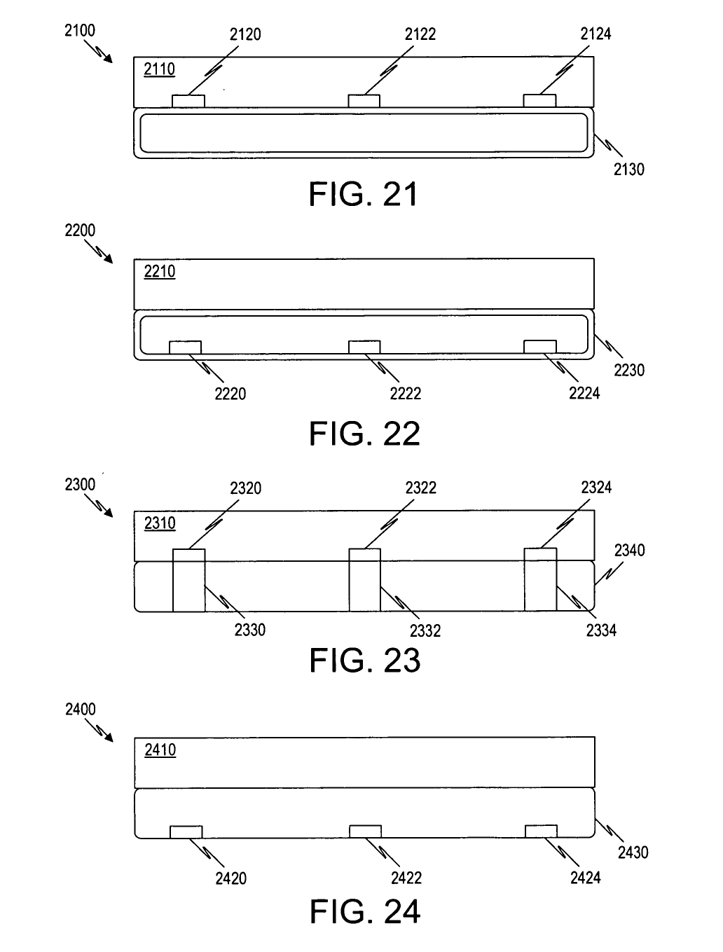

While combining both blood pressure and blood oxygen saturation measurement capability in one sensor is particularly advantageous, the techniques described herein for measuring blood oxygen saturation may be used without blood pressure sensing components. FIG. 21 is a cross-sectional schematic view of an SpO₂ sensor 2100 having a conformable fluid-filled pouch 2130 mounted on a rigid frame 2110. One or more transducers suitable for SpO₂ measurements, illustratively transducers 2120, 2122 and 2124, are mounted on the rigid frame 2110 and positioned to illuminate the surface of a patient's skin through the fluid filled pouch 2130. FIG. 22 is a cross-sectional schematic view of an SpO₂ sensor 2200 having a conformable fluid-filled pouch 2230 mounted on a rigid frame 2210. One or more transducers suitable for SpO₂ measurements, illustratively transducers 2220, 2222 and 2224, are mounted to a diaphragm at the bottom of the pouch 2230, and are positioned to illuminate the surface of a patient's skin through the diaphragm. FIG. 23 is a cross-sectional schematic view of an SpO₂ sensor 2300 having a compressible body 2340 such as foam mounted on a rigid frame 2310. One or more transducers suitable for SpO₂ measurements, illustratively transducers 2320, 2322 and 2324, are mounted on the rigid frame 2310 and positioned to illuminate the surface of a patient's skin through respective optically transparent channels 2330, 2332 and 2334 in the compressible body 2340. FIG. 24 is a cross-sectional schematic view of an SpO₂ sensor 2400 having a compressible body 2430 such as foam mounted on a rigid frame 2410. One or more transducers suitable for SpO₂ measurements, illustratively transducers 2420, 2422 and 2424, are mounted in the compressible body 2430, and are positioned to illuminate the surface of a patient's skin from the bottom thereof. A variety of different shapes and combinations of comformable and/or compressible materials may be used.

It will be appreciated that the sensor in the sensor unit may be unitary, or various components of the sensor may be distributed elsewhere in the sensor unit. Where the sensor includes a pressure transducer, for example, the pressure transducer may be mounted to a supporting member of the sensor that also supports the pressure transmission medium containing the sensing surface, or may be mounted to a supporting member elsewhere in the device and placed in fluid communication with the sensing surface through a fluid-filled tube.

It will be appreciated that although the articulated placement guide is described herein in the context of a wrist-mounted monitoring device, the monitoring device and the associated articulated placement guide may be designed for use with other anatomical structures on which noninvasive monitoring for blood pressure may be performed over a broad range of patient sizes, including children, the elderly, adults, and morbidly obese patients. Such anatomical structures include the inside elbow, the ankle, and the top of the foot.

The description of the invention and its applications as set forth herein is illustrative and is not intended to limit the scope of the invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A noninvasive sensor for use on an anatomical structure of a patient to obtain at least one vital sign, comprising:
a supportive body;
a conformable body coupled to the supportive body and having a contact surface for contacting the anatomical structure;
an optical window disposed at the contact surface; and
a refraction-mode optical transducer sensitive to arterial oxyhemoglobin saturation, the optical transducer being optically coupled to the optical window.

2. The noninvasive sensor of claim 1 wherein:
the conformable body comprises a generally disk-shaped body of compressible material, the contact surface being one of the major surfaces of the disk-shaped body; and
the optical transducer and the optical window are integrated into a unitary device that is mounted in the compressible material.

3. The noninvasive sensor of claim 1 further comprising:
a pressure-transmissive medium having a surface disposed at the contact surface; and
a pressure transducer coupled to the pressure-transmissive medium for sensing pressure therein.

4. The noninvasive sensor of claim 3 wherein:
the pressure-transmissive medium is conformable; and
the conformable body further comprises a generally annular body coupled to the supportive body and generally encircling the pressure-transmissive medium.

5. The noninvasive sensor of claim 4 wherein the generally annular body comprises:
a conformable ring mounted to the supporting body; and
a compressible ring mounted to the conformable ring.

6. The noninvasive sensor of claim 5 wherein:
the pressure-transmissive medium comprises a fluid-filled pouch; and
the conformable ring comprises a fluid-filled ring.

7. The noninvasive sensor of claim 4 further comprising:
a transducer housing mounted on the pressure-transmissive medium;
wherein the pressure transducer and the optical transducer are mounted in the transducer housing;
wherein the pressure-transmissive medium is optically non-opaque; and
the optical transducer is oriented with a field of view through the pressure-transmissive medium and at least a portion of the contact surface, the contact surface portion being non-opaque to form the optical window.

8. The noninvasive sensor of claim 4 wherein:
the optical transducer is mounted in the supportive body;
the pressure-transmissive medium is optically non-opaque; and
the optical transducer is oriented with a field of view through the pressure-transmissive medium and at least a portion of the contact surface, the contact surface portion being non-opaque to form the optical window.

9. The noninvasive sensor of claim 4 wherein:
the optical transducer is mounted within the pressure-transmissive medium; and
the optical transducer is oriented with a field of view through at least a portion of the contact surface, the contact surface portion being non-opaque to form the optical window.

10. The noninvasive sensor of claim 9 wherein the optical transducer is mounted within the pressure-transmissive medium in proximity to the contact surface.

11. The noninvasive sensor of claim 9 wherein:
the optical transducer is mounted away from the contact surface; and
the pressure-transmissive medium is optically non-opaque.

12. The noninvasive sensor of claim 4 wherein:
the optical transducer is mounted in the supportive body proximate to a contact region of the annular body with the supportive body;
the annular body comprises an optically non-opaque channel; and
the optical transducer is oriented with a field of view through the channel in the annular body and through at least a portion of the contact surface, the contact surface portion being non-opaque to form the optical window.

13. The noninvasive sensor of claim 4 wherein:
the optical transducer is mounted within the annular body in proximity to the contact surface; and
the optical transducer is oriented with a field of view through at least a portion of the contact surface, the contact surface portion being non-opaque to form the optical window.

14. The noninvasive sensor of claim 13 wherein the optical transducer is mounted generally at a surface of the annular body.

15. The noninvasive sensor of claim 13 wherein:
the annular body comprises an optically non-opaque channel; and
the optical transducer is mounted away from any surface of the annular body and is oriented with a field of view through the channel in the annular body and at least a portion of the contact surface, the contact surface portion being non-opaque to form the optical window.

16. A noninvasive sensor for use on an anatomical structure of a patient to obtain at least one vital sign, comprising:
a generally disk-like supportive body;
a generally disk-like conformable body coupled to the supportive body and having a contact surface for contacting the anatomical structure, the conformable body comprising:
a generally conformable pressure-transmissive medium comprising a fluid-filled pouch having a surface disposed at the contact surface;
a generally annular conformable body having a first surface coupled to the supportive body, and a second surface opposite the first surface, the annular conformable body generally encircling the conformable pressure-transmissive medium; and
a generally annular compressive body comprising pressure-attenuating material, the annular compressive body having a first surface abutting the second surface of the annular conformable body, and a second surface disposed at the contact surface, the annular compressive body generally encircling the conformable pressure-transmissive medium;
an optical window disposed at the contact surface;
a refraction-mode optical transducer sensitive to arterial oxyhemoglobin saturation, the optical transducer being optically coupled to the optical window; and
a pressure transducer coupled to the pressure-transmissive medium.

17. The noninvasive sensor of claim 16 further comprising:
a transducer housing mounted on the fluid-filled pouch;
wherein the pressure transducer and the optical transducer are mounted in the transducer housing;
wherein the fluid-filled pouch is optically non-opaque; and
the optical transducer is oriented with a field of view through the fluid-filled pouch and at least a portion of the contact surface, the contact surface portion being non-opaque to form the optical window.

18. The noninvasive sensor of claim 16 wherein:
the optical transducer is mounted in the supportive body;
the fluid-filled pouch is optically non-opaque; and
the optical transducer is oriented with a field of view through the fluid-filled pouch and at least a portion of the contact surface, the contact surface portion being non-opaque to form the optical window.

19. The noninvasive sensor of claim 16 wherein:
the optical transducer is mounted within the fluid-filled pouch; and
the optical transducer is oriented with a field of view through at least a portion of the contact surface, the contact surface portion being non-opaque to form the optical window.

20. The noninvasive sensor of claim 19 wherein the optical transducer is mounted within the fluid-filled pouch in proximity to the contact surface.

21. The noninvasive sensor of claim 19 wherein:
the optical transducer is mounted away from the contact surface; and
the fluid-filled pouch is optically non-opaque.

22. The noninvasive sensor of claim 16 wherein:
the optical transducer is mounted in the supportive body proximate to a contact region of the annular conformable body with the supportive body;
the annual conformable body is non-opaque;
the annular compressive body comprises an optically non-opaque channel; and
the optical transducer is oriented with a field of view through the annual conformable body, through the channel in the annular compressive body, and through at least a portion of the contact surface, the contact surface portion being non-opaque to form the optical window.

23. The noninvasive sensor of claim 16 wherein:
the optical transducer is mounted within the annular compressive body in proximity to the contact surface; and
the optical transducer is oriented with a field of view through at least a portion of the contact surface, the contact surface portion being non-opaque to form the optical window.

24. The noninvasive sensor of claim 23 wherein the optical transducer is mounted generally at a surface of the annular compressive body.

25. The noninvasive sensor of claim 23 wherein:
the annular compressive body comprises an optically non-opaque channel; and
the optical transducer is mounted away from any surface of the annular body and is oriented with a field of view through the channel in the annular body and at least a portion of the contact surface, the contact surface portion being non-opaque to form the optical window.

26. A system for use on an anatomical structure of a patient to noninvasively obtain at least one vital sign, comprising:
a generally rigid body;
a hold-down assembly incorporated into the body;
a retainer extending from the rigid body for engaging the anatomical structure upon activation by the hold-down assembly; and
a noninvasive sensor pivotally extending from the body, wherein the noninvasive sensor comprises:
a supportive body;
a conformable body coupled to the supportive body and having a contact surface for contacting the anatomical structure;
an optical window disposed at the contact surface; and
a refraction-mode optical transducer sensitive to arterial oxyhemoglobin saturation, the optical transducer being optically coupled to the optical window.

27. The noninvasive sensor of claim 26 further comprising:
a pressure-transmissive medium having a surface disposed at the contact surface; and
a pressure transducer coupled to the pressure-transmissive medium for sensing pressure therein.

28. The system of claim 27 wherein the retainer comprises a strap, the anatomical structure being a human wrist, further comprising a placement guide extending from the body for guiding placement of the sensor upon the distal edge of the radius bone.

29. The system of claim 28 wherein the placement guide is an articulated placement guide suitable for wrist sizes over a range of about 11 cm to about 22 cm.

30. The system of claim 27 further comprising a control unit mounted on the generally rigid body, the control unit being electrically coupled to the hold-down assembly for controlling hold down pressure, and electrically coupled to the pressure transducer and to the optical transducer for receiving signals therefrom and for calculating blood pressure and oxyhemoglobin saturation.

31. The system of claim 27 further comprising a control unit remote from the rigid body, the control unit being electrically coupled to the hold-down assembly for controlling hold down pressure, and electrically coupled to the pressure transducer and to the optical transducer for receiving signals therefrom and for calculating blood pressure and oxyhemoglobin saturation.

32. The system of claim 27 further comprising a control unit electrically coupled to the hold-down assembly for controlling hold down pressure, and electrically coupled to the pressure transducer and to the optical transducer for receiving signals therefrom and for calculating and displaying blood pressure and oxyhemoglobin saturation.

33. The system of claim 32 further comprising a tympanic-type temperature sensor, the control unit being electrically coupled to the temperature sensor for receiving signals therefrom indicative and for calculating and displaying temperature.
